(19) United States
(12) Patent Application Publication
(10) Pub. No.: US 2024/0164881 A1
(43) Pub. Date: May 23, 2024

(54) HIGH VOLUME EVACUATOR WITH MIRROR SECUREMENT

(71) Applicant: Thad Pierce Johnston, Gastonia, NC (US)

(72) Inventors: James Christopher Stango, Chicago, IL (US); Thad Pierce Johnston, Gastonia, NC (US)

(21) Appl. No.: 18/379,012

(22) Filed: Oct. 11, 2023

Related U.S. Application Data
(60) Provisional application No. 63/426,113, filed on Nov. 17, 2022.

(51) Int. Cl. A61C 17/06; A61B 1/247

(52) U.S. Cl. CPC A61C 17/088; A61B 1/247

(57) ABSTRACT

A high volume evacuator (HVE) device, for use in dental procedures, securely receives a threaded stem of a reusable dental mirror. The housing of the HVE device includes an axially-extending depression having a pair of parallel sidewalls, each of the sidewalls having a plurality of ribs oriented in a direction perpendicular to an axis of the housing. The ribs serve to retain threads of a threaded stem of a dental mirror. A passthrough opening is provided to permit the threaded stem of the dental mirror to pass from an interior of the housing of the HVE device into the axially-extending depression. The housing of the HVE device includes structural members that facilitate insertion of a dental mirror in a manner that direct the threaded stem toward and through the passthrough opening. The housing of the HVE device may also include intake holes to draw in aerosols other than through a mouth at a distal end of the HVE device.

### CROSS-REFERENCE TO RELATED APPLICATION

This disclosure claims the benefit of the filing date of U.S. Provisional Application No. 63/426,113, filed Nov. 17, 2022, the entirety of which is hereby incorporated by reference.

### FIELD OF THE DISCLOSURE

This disclosure relates generally to suction devices for use in oral cavity procedures and, more specifically, to a high volume evacuator that provides secure retention of a dental mirror.

### DESCRIPTION OF THE PRIOR ART

Past improvements to high volume evacuator (HVE) devices used in dental procedures included structurally associating dental mirrors with HVE devices. The dental mirror was often a stemless disc shape, and secured within a complementary mouth, or aperture, at a distal end of the HVE device in a manner that permitted air and suctioned material to pass between the mirror and the mouth of the HVE. In other prior approaches to associating dental mirrors with HVE devices, a stem of the dental mirror was threadedly-secured in a piggyback fashion, entirely externally of the main passageway defined by the HVE housing. According to yet further approaches to associating dental mirrors with HVE devices, a mirror is clipped to a mouth portion of an HVE device.

It would be desirable to provide an HVE device that structurally associates a dental mirror in a manner that provides clearance between a mouth of the HVE device and the dental mirror around an entire circumference of the mirror, while providing a manner of attachment that masks at least a majority of a length of the mirror stem so as to avoid the stem causing any obstructions in the oral cavity during a dental treatment procedure.

### SUMMARY OF THE DISCLOSURE

An HVE device of the present disclosure includes a main, hollow body having a mouth at a first end thereof, the mouth preferably shaped and dimensioned so as to receive a round dental mirror in a manner that minimizes obstruction to visibility of the reflective surface(s) of the mirror and provides clearance between the mouth of the HVE device and the entire circumference of the dental mirror. The HVE device shares features in common with the HVE devices disclosed in international patent application PCT/US2021/032191, filed May 13, 2021, and published Nov. 18, 2021, as WO 2021/231696 A1, the US national phase of which was entered Nov. 10, 2022 and accorded U.S. patent application Ser. No. 17/924,463, the entirety of each of which is hereby incorporated by reference. The HVE device has a second end opposite the first end, which second end is attachable, via suction tubing, to a negative pressure source. The body of the HVE device defines a primary conduit extending between the first and second ends. The body of the HVE device is provided with an axially-extending depression including parallel sidewalls. Each of the parallel sidewalls includes a plurality of spaced laterally-extending ribs projecting outwardly therefrom, i.e., each of the ribs extends in a direction perpendicular to the axis of the body of the HVE device and projects outwardly (relative to an interior of the body of the HVE device) from the surface of the sidewall. Each of the laterally-extending ribs is aligned with a corresponding laterally-extending rib that projects outwardly from the respective parallel sidewall in a direction toward the opposite sidewall. The laterally-extending ribs are preferably spaced so as to mimic a threaded female connection for a standard #4-48 mouth mirror thread, such as the ⅞ in (22 mm) diameter double-sided cone socket mirror available from Hu-FriedyGroup, a STERIS company.

The HVE device facilitates the temporary connection of a mouth mirror for the purposes of extraction of aerosols and other fine particles during dental procedures, while providing low-obstructed visibility during dental procedures via the dental mirror. For use, a dental mirror is inserted into the mouth end of the body of the HVE device, with the male-threaded shaft of the dental mirror inserted into a preferably rectangular-shaped keyway opening region provided in the body, through (e.g., the male-threaded shaft moving through via translational or linear motion) a passage or hole at a base of the keyway opening region that opens to a top of the axially-extending depression, and the head of the dental mirror is pushed toward the second end of the body of the HVE device until the male-threaded shaft of the dental mirror is pressed into (e.g., moves into via translational or linear motion) the receptacle defined by the axially-extending depression with integrated laterally-extending ribs. The parallel sidewalls of the axially-extending depression are deformable (such as by employing the deformable properties of injection molded polymeric material used to mold the HVE device), permitting the insertion of the male-threaded shaft of the dental mirror past (e.g., the male-threaded shaft moves past via translational or linear motion) pairs of laterally-extending ribs until a portion of the male threads pass at least one position indicator line provided on the exterior of the body of the HVE device. The insertion force required is less than 10 pounds, and the HVE device retains the dental mirror within the receptacle during use with a minimum extraction force of 5 pounds, thereby preventing inadvertent ejection of the dental mirror from the HVE device during use. Once use is completed, the dental mirror can be expelled from the receptacle by pulling on the head of the mirror with a force in excess of the minimum extraction force, after which the dental mirror can be sanitized (such as in an autoclave) for re-use and the HVE device can be discarded.

### BRIEF DESCRIPTION OF THE SEVERAL VIEWS OF THE DRAWING

FIG. 1 is a rear, left, top perspective view of a high volume evacuation (HVE) device of the present disclosure;

FIG. 2 is a front, left, top perspective view of the HVE device of the present disclosure;

FIG. 3 is a right side view of the HVE device of the present disclosure;

FIG. 4 is a rear view of the HVE device of the present disclosure;

FIG. 5 is a left side view of the HVE device of the present disclosure;

FIG. 6 is a rear, left, bottom perspective view of the HVE device of the present disclosure;

FIG. 7 is a front view of the HVE device of the present disclosure;

FIG. 8 is a bottom view of the HVE device of the present disclosure;

FIG. 9 is a top view of the HVE device of the present disclosure;

FIG. 10 is a front, left, top perspective, cross-section view taken along lines 10-10 of FIG. 7;

FIG. 11 is a front, left, top perspective, cross-section view taken along lines 11-11 of FIG. 7;

FIG. 12 is a rear, left, top perspective, cross-section view taken along lines 12-12 of FIG. 3;

FIG. 13 is a front, left, top perspective, cross-section view taken along lines 13-13 of FIG. 4;

FIG. 14 is an enlarged vertical cross-section illustrating a front of the mouth, a keyway opening, and a passthrough opening, of the HVE device of FIG. 1;

FIG. 15 is a vertical cross-section view taken along lines 15-15 of FIG. 7;

FIG. 16 is a perspective view of a conventional dental mirror having a threaded stem;

FIG. 17 is a perspective view of another HVE device of the present disclosure in combination with the dental mirror of FIG. 15;

FIG. 18 is a right side view of the HVE device and mirror combination illustrated in FIG. 17;

FIG. 19 is a front view of the HVE device and mirror combination illustrated in FIG. 17;

FIG. 20 is a bottom, front, right perspective view of the HVE device and mirror combination illustrated in FIG. 17;

FIG. 21 is an enlarged front, left perspective view of the portion of the HVE device and mirror combination illustrated in FIG. 20 encircled by the line designated as 21 in FIG. 20;

FIG. 22 is a vertical cross-section of the HVE device and mirror combination illustrated in FIG. 17, taken along the lines 22-22 in FIG. 18;

FIG. 23 is a top view of the HVE device and mirror combination illustrated in FIG. 17;

FIG. 24 is a bottom view of the HVE device and mirror combination illustrated in FIG. 17; and

FIG. 25 is a front view of yet another HVE device of the present disclosure, in combination with a dental mirror.

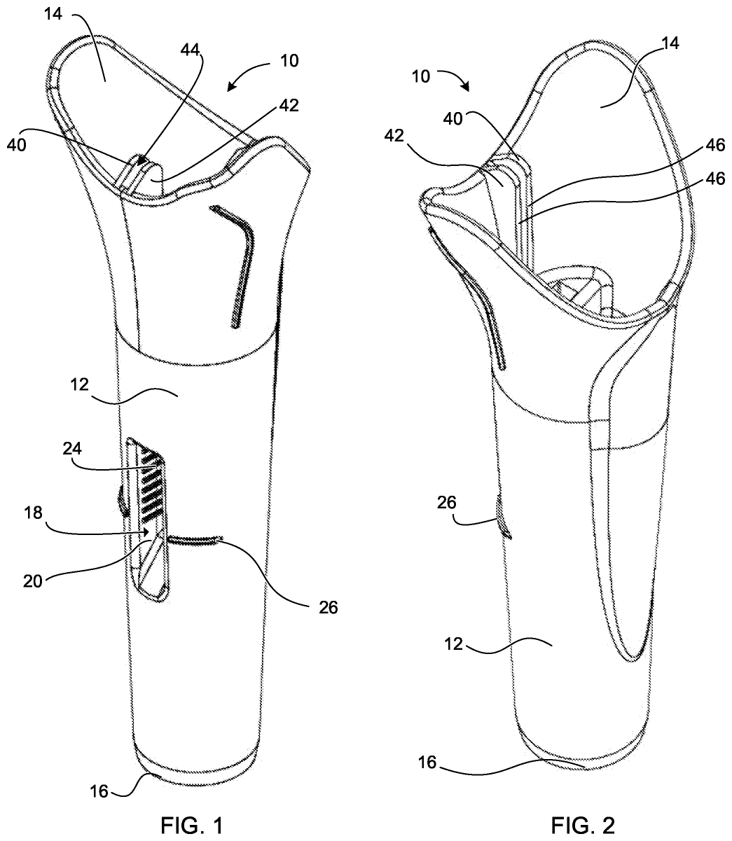

### DETAILED DESCRIPTION OF THE PREFERRED EMBODIMENTS

A high volume evacuator (HVE) device 10 of the present disclosure includes a housing 12 (e.g., a hollow tubular body) defining a primary conduit extending between a first end 14 including a mouth and a second end 16 opposite the first end 14. An axially-extending depression 18 (e.g., an elongate opening, alcove, hole, or receptacle) is provided in a rear of the housing 12. The axially-extending depression 18 is defined by a pair of sidewalls 20, 22. An inclined ramp portion 23 may be provided at a lowermost end of the axially-extending depression 18, i.e. at an end of the axially-extending depression 18 closest to the second end 16 of the housing 12.

Each of the sidewalls 20, 22 is provided with a plurality of parallel ribs 24, each rib 24 disposed perpendicular to a longitudinal axis of the housing 12 of the HVE device 10. Each of the ribs 24 of the plurality of parallel ribs provided along the first sidewall 20 is vertically offset from each of the ribs of the plurality of parallel ribs provided along the second sidewall 22. On an exterior of the housing 12, at least one line is provided, which may be in the form of a raised indicator rib 26, oriented parallel to each of the ribs 24. The indicator rib 26 serves as a locator to inform a user when the threads along the stem of a dental mirror have been sufficiently inserted into the axially-extending depression 18 so as to securely retain the dental mirror within the HVE device.

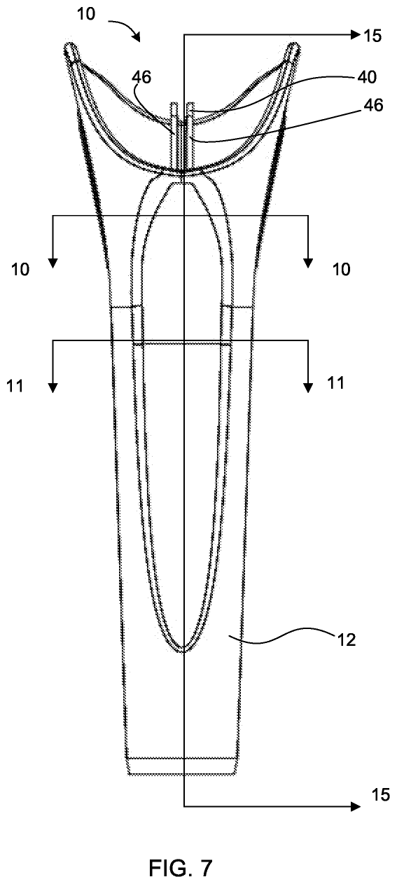

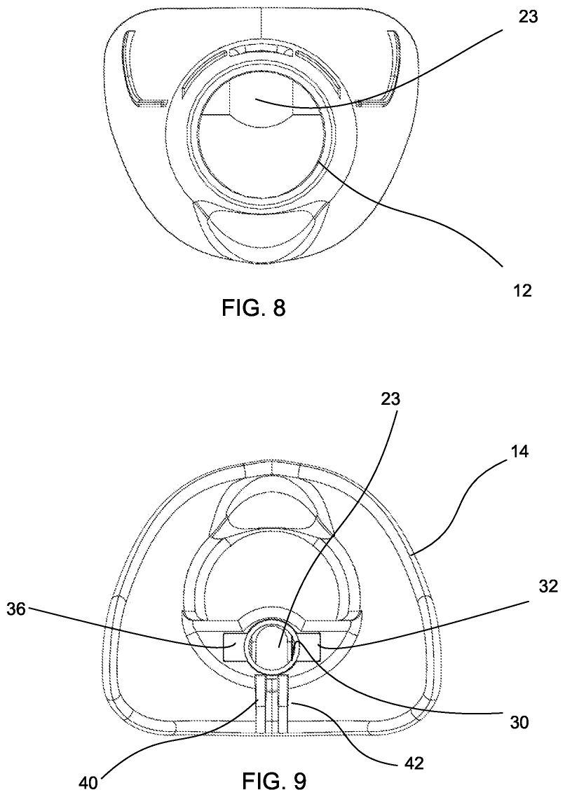

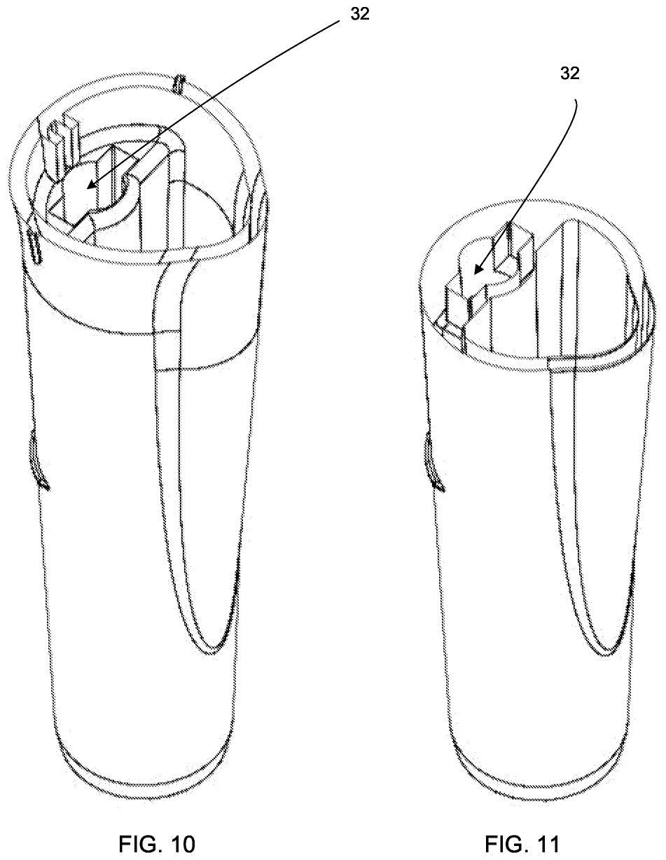

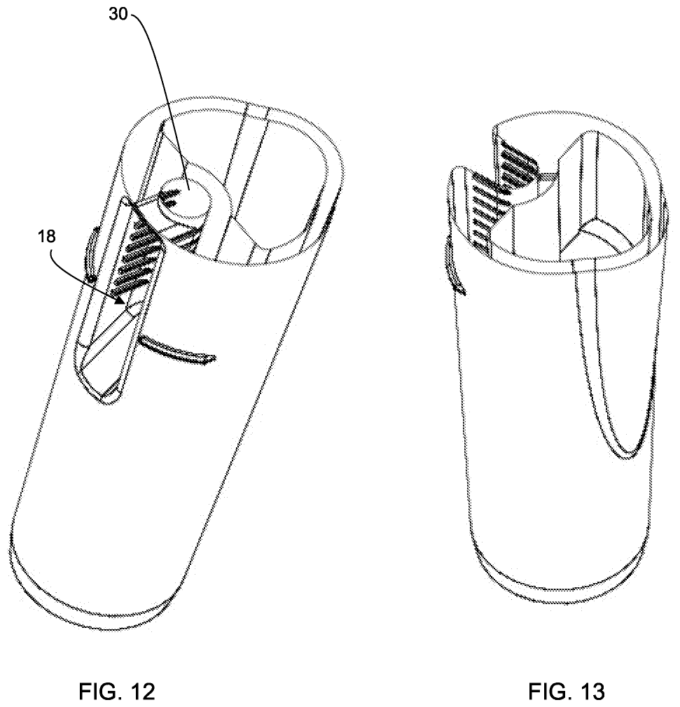

Turning to FIG. 6, at the top of the axially-extending depression 18, i.e. at an end of the axially-extending depression 18 closest to the first end 14 of the housing 12, a passthrough aperture 30 (e.g., an opening, hole, or receptacle) is provided. This passthrough aperture 30 accommodates a threaded stem of a dental mirror (as explained in more detail below) to pass from an interior of the housing 12 of the HVE device 10 to the axially-extending depression 18.

The sidewalls 20, 22 are separated from one another an appropriate distance, and the spacing of the ribs 24 along each of the respective sidewalls 20, 22 is such that the ribs 24 complement the external threads along the stem of a dental mirror, serving as mating or engaging structure (e.g., internal threads), so as to promote secure engagement (e.g., interference fit) between the dental mirror and the axially-extending depression 18 of the housing of the HVE device. The HVE device may be formed of a material having an elasticity such that the ribbed sidewalls 20, 22 of the axially-extending depression 18 deform outwardly as the stem of a dental mirror is pressed into the axially-extending depression 18 a sufficient extent so as to permit the external threads along the stem of a dental mirror to advance past the ribs 24 along the first and second sidewalls 20, 22 of the axially-extending depression 18, and then immediately rebound.

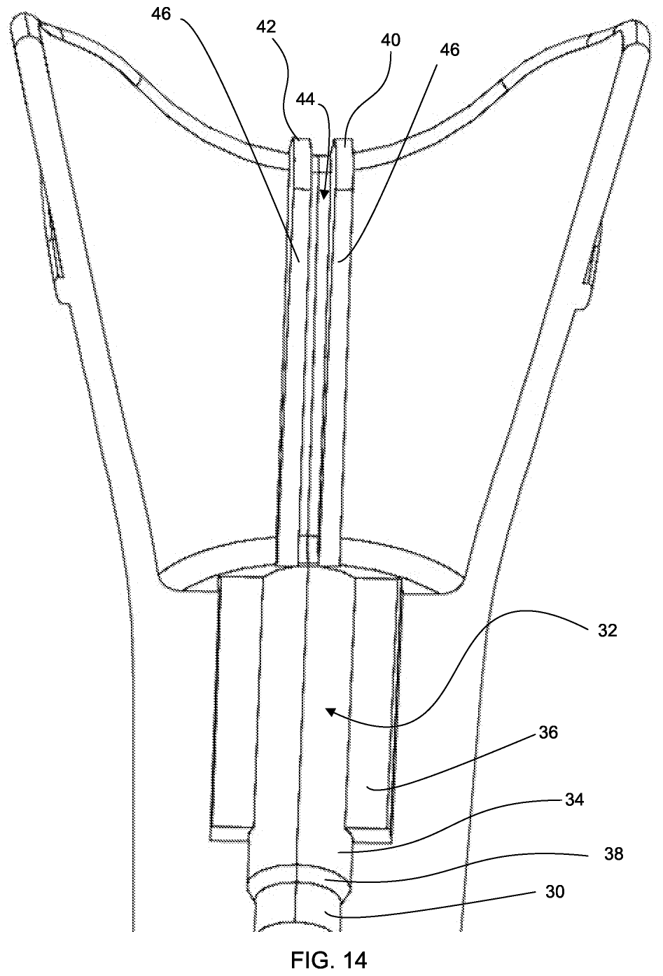

Above the passthrough aperture 30, which is circular in cross-section, a keyway opening 32 (e.g., a guideway, receptacle, or passageway) may be provided. The keyway opening 32 is preferably generally shaped in the form of a tubular region 34 that is coaxial with the passthrough aperture 30, which tubular region 34 is bisected by a rectangular-shaped opening 36, the longer dimension (in cross-section) of the rectangular-shaped opening 36 extending in a direction parallel to a front of the HVE device 10. The diameter of the tubular region 34 is greater than the diameter of the passthrough aperture 30. This keyway opening 32 accommodates a flat support region of the stem of a dental mirror that may extend between a threaded region of the stem and a rounded portion of the dental mirror having one or two reflective surfaces. As illustrated in FIG. 14, a base 38 of the tubular region 34, adjacent the passthrough aperture 30, the wall of the tubular region 34 tapers inwardly to the diameter of the passthrough aperture 30, which serves to help direct a lower threaded portion of the stem toward and through the passthrough aperture 30 as a user pushes the stem of a dental mirror down through the keyway opening 32 in an effort to insert the stem through the passthrough aperture 30 so the threads of the stem can engage the ribs 24 along the first and second sidewalls 20, 22 of the axially-extending depression 18.

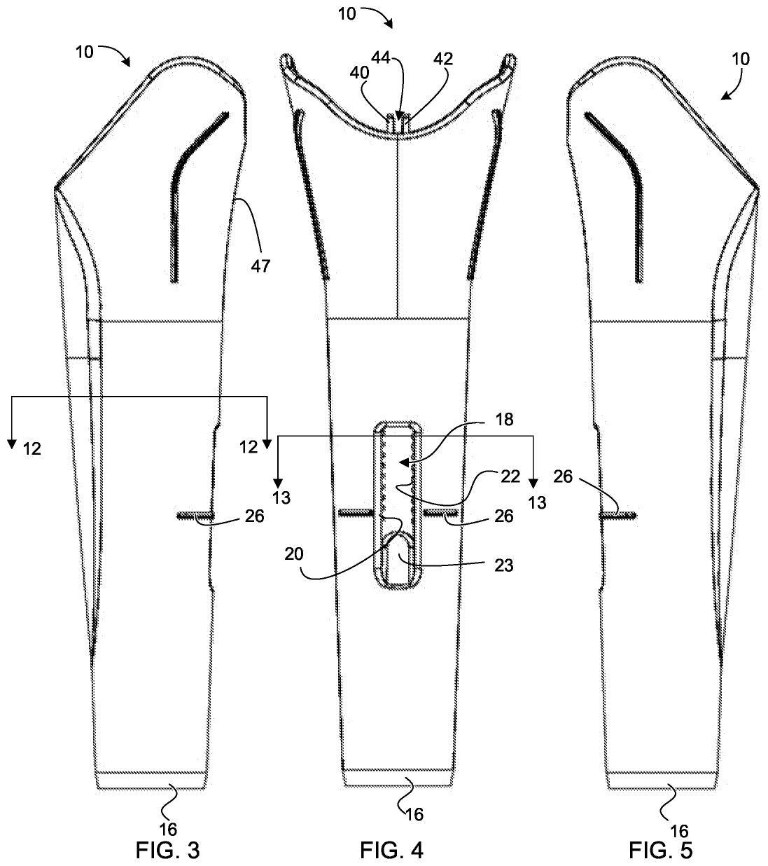

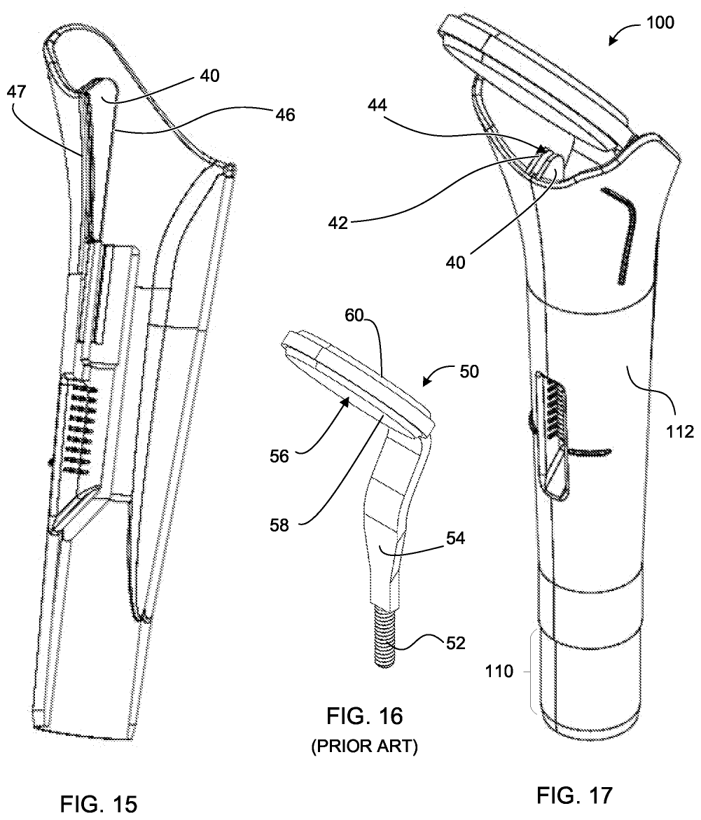

Along a front of the housing 12, on an inside of the primary conduit defined by the housing 12, a pair of axially-extending stanchions 40, 42 is provided. The stanchions 40, 42 define a slot 44 between them. Each of the stanchions 40, 42 has a rear edge 46 that extends vertically, parallel to a front wall of the rectangular-shaped opening 36, extending from the mouth at the first end 14 of the HVE device 10 and terminating at an upper edge of the tubular region 34 of the keyway opening 32. While the housing 12 tapers outwardly from the second end 16 toward the first end 14, the slope of the outward taper increases along the front of the housing 12 for the extent of the stanchions 40, 42, as illustrated along the region identified by reference numeral 47 in FIGS. 3 and 15. The rear edges 46 of the stanchions 40, 42 cooperate with one another to define a guide plane to direct a flat support region of the stem of a dental mirror down and into the keyway opening 32 to facilitate insertion of a threaded end of the stem of the dental mirror through the passthrough aperture 30, outside the housing 12, and into the axially-extending depression 18 for securement between the ribs 24 along the sidewalls 20, 22 of the depression 18.

An HVE device 10 of the present disclosure is preferably a disposable device, but is intended to be paired for use with a standard reusable mouth mirror, such as the dental mirror 50 (e.g., dental mouth mirror apparatus) illustrated in FIG. 16. The dental mirror 50 includes a threaded stem 52. A flat support region 54 extends between the threaded stem 52 and a circular mirror end 56, which includes a reflective mirror surface 58 on a front side, and may additionally include a second mirror surface 60 on a rear side.

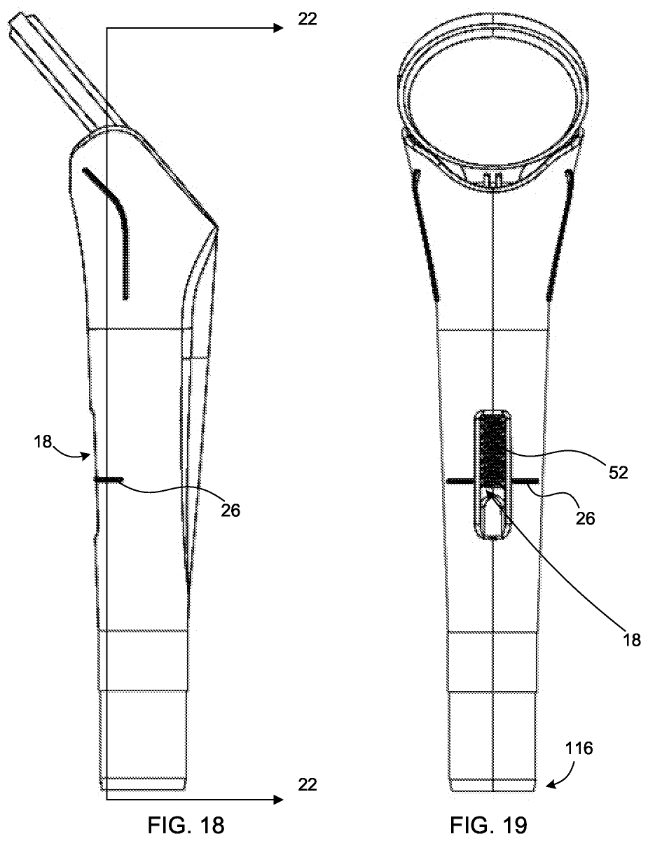

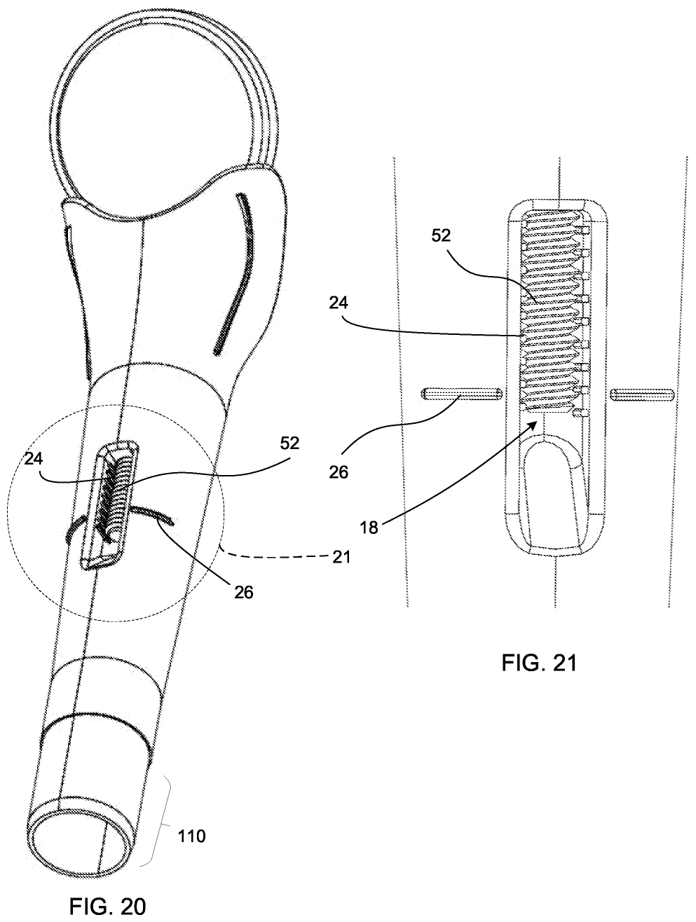

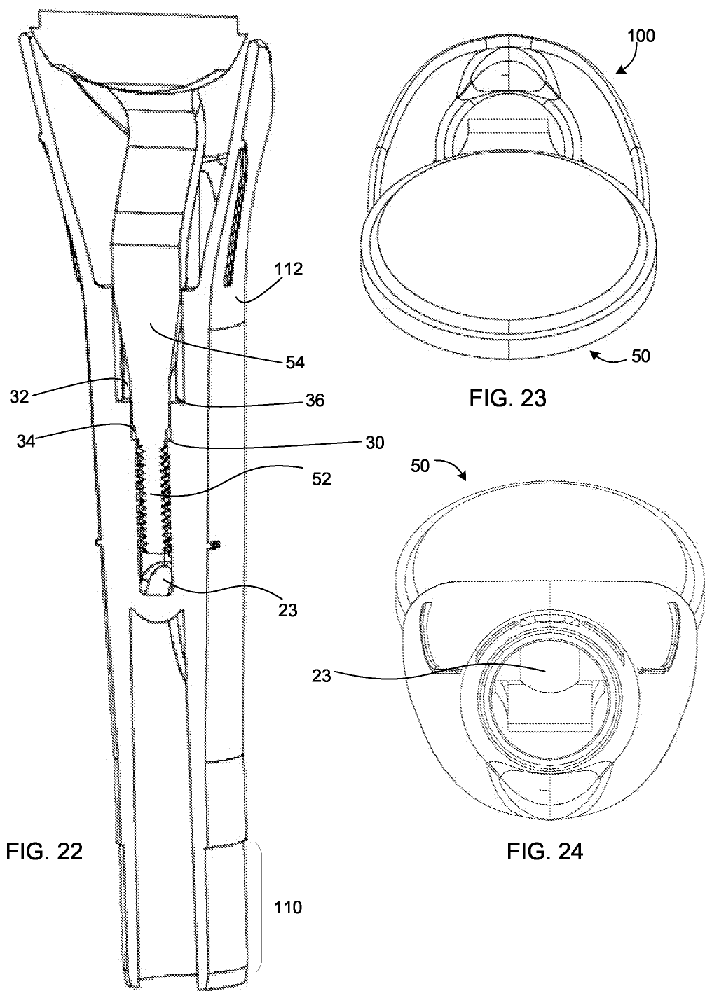

Another HVE device 100 of the present disclosure is illustrated in FIGS. 17-24. The HVE device 100 shares many features in common with the HVE device 10, with all common features that are designated by reference number being designated with the same reference numbers as employed above with respect to that HVE device 10. A housing 112 of the HVE device 100 is provided with an inwardly-stepped cylindrical handpiece engagement region 110 at a second end 116 thereof. The inwardly-stepped cylindrical handpiece engagement region 110 provides an enhanced fit with a mating handpiece, with the annular step serving as a positive stop when the HVE device 110 is fully inserted into a distal end of the handpiece.

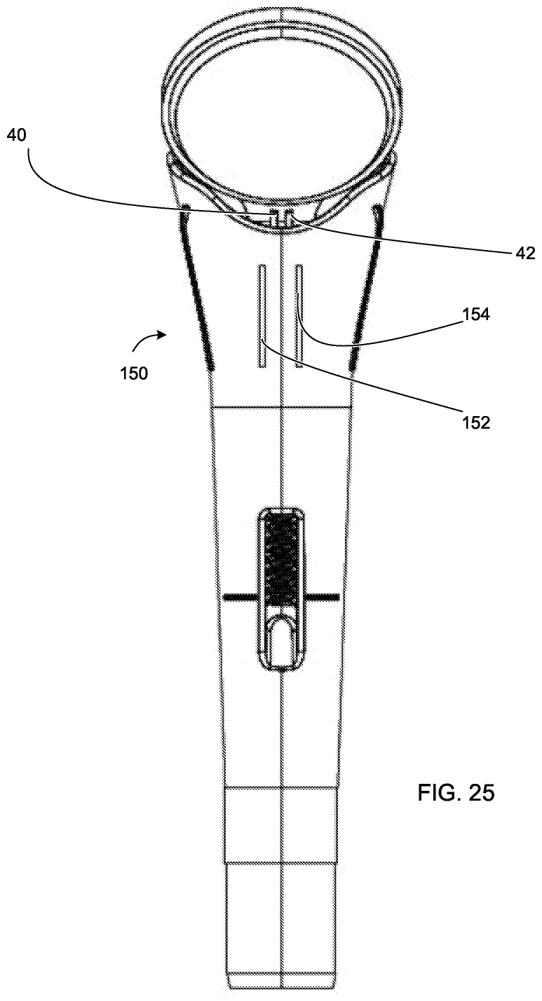

Yet another HVE device 150 of the present disclosure is illustrated in FIG. 25. The HVE device according to this embodiment also shares many features in common with the HVE devices 10, 100, with all common features that are designated by reference number being designated with the same reference numbers as employed above with respect to those HVE devices 10, 100. The HVE device 150 is provided with intake holes 152, 154, which may be in the form of slots, which may extend along the length of the housing 12. While two intake holes 152, 154 are illustrated, and are illustrated as being rectangular in shape, fewer or more intake holes can be provided, and the intake hole(s) may have any suitable shape, such as square, round, oblong, or elliptical. Each of the intake holes 152, 154 is preferably provided laterally outboard of the stanchions 40, 42.

The intake holes 152, 154 can be positioned sufficiently close to a first, distal end of the HVE device 150 such that, during use, at least aerosols from within the dental patient's mouth can be drawn into the intake holes 152, 154. Alternately or additionally, one or more such intake holes 152, 154 can be positioned relatively outwardly from the patient's mouth, i.e. along a rear side of the housing of the HVE device 150, for intaking at least aerosols that may have exited the patient's mouth.

As an example at least partially alluded to above, in addition to or as an alternative to using the above-discussed ribs 24, releasable connection(s) (e.g., interference fit(s) provided by mating parts that fit tightly together) may be provided by engagements between other suitable inner surface(s) of the HVE device 10, 100, 150 and respective outer surface(s) of a dental mirror (e.g., the dental mirror 50 or a differently configured dental mouth mirror apparatus). As another example, for ease of use and/or in an effort to have the dental mirror be manually mounted to the HVE device in a predetermined configuration, the respective structural features that are mated together to provide the interference fit(s) (e.g., ribs 24) and/or other structural feature(s) (e.g., keyway opening 32) can be cooperatively configured so that the interference fit(s) are achieved in response to (e.g., at least partially or solely in response to) relative translational or linear motion between the dental mirror and the HVE device (e.g., the dental mirror being pushed into place) while the dental mirror and the HVE device are in a predetermined orientation with respect to one another.

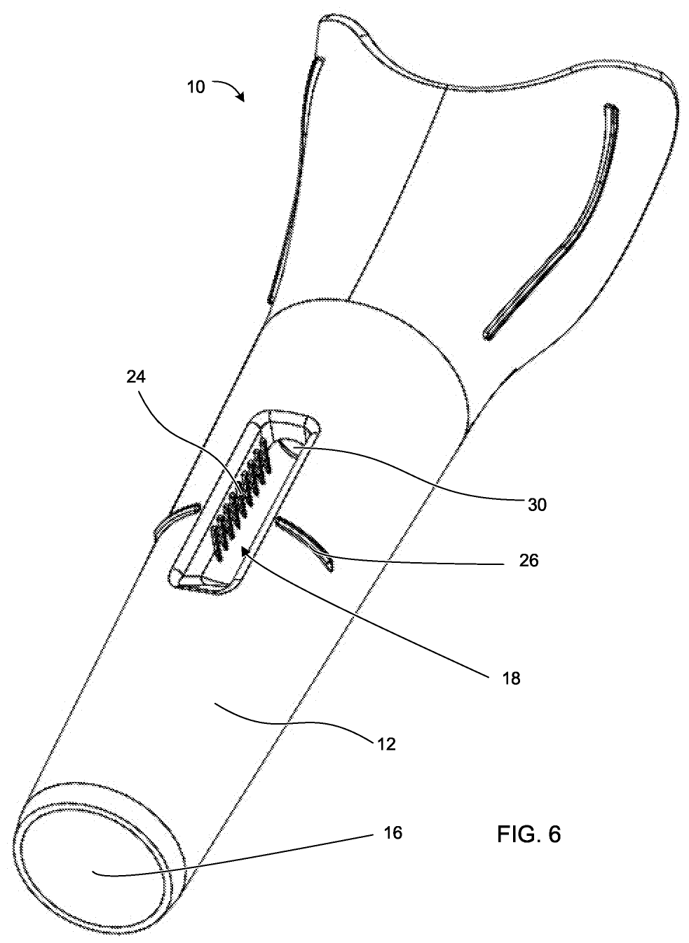

In the examples depicted in the drawings, the HVE device includes an undulating annular end edge defining the first end 14 (see, e.g., FIG. 9) of the HVE device. Referring to FIGS. 3-5 and 7, the end edge defining the first end 14 of the HVE device can define one or more, or more specifically two opposite, concave edge portions (e.g., recessed portions), or the like, extending toward the second end 16 of the HVE device, and the first end edge can further define one or more, or more specifically two opposite, convex edge portions (e.g., protruding portions), or the like, extending away from the second end 16. These convex edge portions can be parts of one or more lobes, tabs, or tab portions of the HVE device that can respectively be proximate and/or engaged against the head of the mirror 50, for example opposite sides of the mirror head as depicted in FIGS. 17-20 and 22-25, as part of an overall stable mounting of the mirror to the HVE device.

While various embodiments have been described herein, it will be understood that variations may be made thereto that are still within the scope of the appended claims.

What is claimed is:

1. A high volume evacuation device adapted to securely retain a dental mirror, comprising:
a housing including
a mouth defining a first end of the housing;
a primary conduit depending downwardly from the mouth;
a mirror stem retention mechanism comprising at least one set of parallel ribs extending transversely to a central axis of the primary conduit.

2. The high volume extraction device of claim 1, wherein the at least one set of parallel ribs is provided on an exterior surface of the primary conduit of the housing.

3. The high volume evacuation device of claim 1, further comprising a secondary conduit extending parallel to the primary conduit along at least a portion of the length of the primary conduit.

4. The high volume evacuation device of claim 3, wherein at least a portion of the length of the secondary conduit has a generally keyway-shaped cross-section, with a round central opening bisected by a rectangular opening.

5. The high volume evacuation device of claim 3, the housing further including an axially-extending depression in an outer wall thereof, the secondary conduit opening into the axially-extending depression.

6. The high volume extraction device of claim 5, wherein the at least one set of parallel ribs includes two sets of parallel ribs, a first of the sets of parallel ribs provided along a first wall of the axially-extending depression in the outer wall of the housing, and a second of the sets of parallel ribs provided along an opposite wall of the axially-extending depression in the outer wall of the housing.

7. The high volume extraction device of claim 6, wherein each of the ribs along the second of the sets of parallel ribs is vertically offset from each of the ribs of the first of the sets of parallel ribs.

8. The high volume extraction device of claim 7, in combination with a dental mirror having a stem with a threaded distal end, the threaded distal end of the stem secured between the first and second sets of parallel ribs.

9. The combination of claim 8, the housing including at least one indicator line on an exterior thereof, the at least one indicator line oriented parallel to the ribs of the first and second sets of ribs, and a distal tip of the stem of the dental mirror extending beyond the indicator line in a direction toward a second end of the housing opposite from the first end of the housing.

10. The combination of claim 8, the at least one indicator line including a first elongate rib disposed to one side of the axially-extending depression and a second elongate rib disposed to an opposite side of the axially-extending depression.

11. A dental evacuation tip configured to be connected to a mirror apparatus and for high-volume evacuation of air and/or aerosols from a dental patient's mouth, the dental evacuation tip comprising:
a tube comprising opposite proximal and distal ends, and at least one sidewall extending around an interior passageway of the tube;
opposite proximal and distal openings to the interior passageway, wherein the distal opening is proximate the distal end, and the proximal opening is proximate the proximal end;
a hole extending through the at least one sidewall; and
opposite tab portions of the at least one sidewall at least partially defining a guideway opening extending from the distal opening to the hole, wherein the hole is wider than the guideway opening;
wherein the dental evacuation tip is configured for releasably securing a mirror apparatus to the tube such that at least a portion of a rod of the mirror apparatus is positioned in the interior passageway and a reflective surface of a mirror of the mirror apparatus is positioned proximate the distal opening;
wherein the dental evacuation tip is configured to be operably connected to a vacuum source such that the dental evacuation tip and the vacuum source are in fluid communication via the proximal opening;
wherein, when the vacuum source is operably connected, the dental evacuation tip is configured to draw air and/or aerosols from a dental patient's mouth such that at least a portion of the air and/or aerosols contact and travel across the reflective surface of the mirror while the mirror apparatus is releasably secured to the tube;
a plurality of holes extending through the at least one sidewall, wherein the plurality of holes are proximate the hole extending through the at least one sidewall, and the plurality of holes are positioned between the hole extending through the at least one sidewall and the proximal end of the tube; and
a mirror stem retention mechanism comprising at least one set of parallel ribs extending transversely to a central axis of the tube.

12. The dental evacuation tip of claim 11, wherein the at least one set of parallel ribs is provided on an exterior surface of the tube.

13. The dental evacuation tip of claim 11, further comprising a secondary conduit extending parallel to the tube along at least a portion of the length of the tube.

14. The dental evacuation tip of claim 13, the tube further including an axially-extending depression in an outer wall thereof, the secondary conduit opening into the axially-extending depression.

15. The dental evacuation tip of claim 14, wherein the at least one set of parallel ribs includes two sets of parallel ribs, a first of the sets of parallel ribs provided along a first wall of the axially-extending depression in the outer wall of the tube, and a second of the sets of parallel ribs provided along an opposite wall of the axially-extending depression in the outer wall of the tube.

16. The dental evacuation tip of claim 15, wherein each of the ribs along the second of the sets of parallel ribs is vertically offset from each of the ribs of the first of the sets of parallel ribs.

17. The dental evacuation tip of claim 15, in combination with a dental mirror having a stem with a threaded distal end, the threaded distal end of the stem secured between the first and second sets of parallel ribs.

18. The combination of claim 17, the housing including at least one indicator line on an exterior thereof, the at least one indicator line oriented parallel to the ribs of the first and second sets of ribs, and a distal tip of the stem of the dental mirror extending beyond the indicator line in a direction toward a second end of the housing opposite from the first end of the housing.

19. The combination of claim 18, the at least one indicator line including a first elongate rib disposed to one side of the axially-extending depression and a second elongate rib disposed to an opposite side of the axially-extending depression.

20. A method for securing a dental mirror in a housing of a high volume evacuation device, comprising:
providing a housing including
a mouth defining a first end of the housing;
a primary conduit depending downwardly from the mouth; and
a mirror stem retention mechanism comprising at least one set of parallel ribs
extending transversely to a central axis of the primary conduit
inserting a stem of the mirror having a threaded distal end into the mirror stem retention mechanism.

21. The method of claim 20, and in providing the housing, the housing further includes a secondary conduit extending parallel to the primary conduit along at least a portion of the length of primary conduit, and inserting the stem of the mirror into the mirror stem retention mechanism includes inserting the stem of the mirror through the secondary conduit.

22. The method of claim 21, and in providing the housing, the housing further includes an axially-extending depression in an outer wall thereof, the secondary conduit opening into the axially-extending depression, and the at least one set of parallel ribs includes two sets of parallel ribs, a first of the sets of parallel ribs provided along a first wall of the axially-extending depression in the outer wall of the housing, and a second of the sets of parallel ribs provided along an opposite wall of the axially-extending depression in the outer wall of the housing, each of the ribs of the first set of parallel ribs being vertically offset from each of the ribs of the second set of parallel ribs; and
inserting the stem of the mirror into the mirror stem retention mechanism includes advancing the mirror until the threaded distal end is secured between the first and second sets of parallel ribs.

23. The method of claim 22, and in inserting the stem of the mirror into the mirror stem retention mechanism, advancing the mirror until a distal tip of the stem of the dental mirror extending beyond an indicator line on an exterior of the housing, the indicator line oriented parallel to the ribs of the first and second sets of ribs, and in a direction toward a second end of the housing opposite from the first end of the housing.
